(19) United States
(12) Patent Application Publication
(10) Pub. No.: US 2003/0006159 A1
(43) Pub. Date: Jan. 9, 2003

(54) Additive tube for enteral nutrition apparatus

(76) Inventors: Jorgen Thorball, Virum (DK); Ivan A. Casas, Raleigh, NC (US)

Correspondence Address: Lynn E. Barber, P.O. Box 16528, Fort Worth, TX, 76162, US

(21) Appl. No.: 09/911,189

(22) Filed: Jul. 23, 2001

Related U.S. Application Data
(63) Continuation-in-part of application No. 09/387,947, filed on Sep. 1, 1999, now Pat. No. 6,283,294.

(51) Int. Cl. A61L 15/00

(52) U.S. Cl. 206/438

(57) ABSTRACT

An additive tube containing a selected material, such as bacterial cells or other additive, for addition a liquid enteral nutrition product dispensed from an enteral bag or bottle. Additive tubes are separately packaged to reduce exposure of the additive that is within the additive tube to water and oxygen. Adapters on each end of the additive tube enable attachment of the additive tube to tubing of the enteral nutrition system (the delivery line, other tubes, and enteral bag or bottle) just prior to use with a patient.

### CROSS-REFERENCE TO RELATED APPLICATIONS

This application is a continuation-in-part of co-pending Ser. No. 09/387,947, filed Sep. 1, 1999.

### BACKGROUND OF THE INVENTION

1. Field of the Invention

This invention relates to device comprising an additive tube used to add a sensitive component to liquid enteral nutrition products by adding the sensitive component during the feeding of the enteral nutrition product into the gastrointestinal tract of the patient.

2. Description of the Related Art

Liquid enteral nutrition products are normally fed to patients from a bottle or bag that is hung near the patient. The liquid typically drips from the bottle or bag into a drip chamber, and then flows by gravity through a flexible tube that leads either to a nasogastric tube extending through a nasal passage and the esophagus to the stomach, or to a feeding tube extending through the abdominal wall to the stomach or small intestine. The nasogastric tube or feeding tube is typically attached to one or more connected pieces of tubing leading from the drip chamber, by means of a connector element or adapter.

In the medical industry it is well-known that the addition of various components to the enteral nutrition system, including sensitive health-promoting products, such as bacteria (e.g. probiotic bacteria such as lactic bacteria or bifido bacteria) allows people to maintain a proper gut function. For over 20 years, the producers of enteral nutrition-tube feeding products have had particular problems, however, with adding unstable components, including oxygen-sensitive and water-sensitive components, such as vitamins, trace elements, and in recent years, probiotic bacteria, to the enteral product. This is due both to thermal processing when producing the enteral products and break down of certain components during storage of these products.

To try to solve the general problem of adding components to an enteral nutrition apparatus, additional chambers have been used within the enteral bag to contain the sensitive additives (e.g., a capsule from which the component can be ejected upon osmotic infusion of moisture into the capsule), or an additional injection port has been made on the bag. Both designs have been difficult to produce and to sterilize. An additional chamber or connector has also been tried between the enteral bag and the infusion spike; however, the spike used for penetrating the closing system of the bag hinders any flow through an upper line connector. For some additives, it has been necessary to overdose the system with the additive due to degradation of the additive in solution, while with other additive components it is often difficult to determine whether the additive is in solution in the proper amount. Other additives influence solution stability, or cannot be added in advance to the solution at all. One way to avoid the above problems has been to take a syringe and manually inject the component, for example, probiotic bacteria suspended in milk, into the enteral bag immediately before usage. This is impractical, unsafe and labor-requiring, cannot be done with hard bottles, and in practice, hinders a predetermined release rate.

The patent of Walton et al. (U.S. Pat. No. 5,738,651) provides an apparatus for altering the composition of liquid nutritional products with additives during enteral tube feeding. The apparatus utilizes a drip-chamber or other formulation chamber, into which a useful amount of at least one beneficial agent in controlled release dosage form is added, with or without a marker dye. The disclosure of this patent and of all other patents and publications referred to herein is incorporated herein by reference.

With respect to additives comprising living microorganisms, it has been difficult and relatively expensive to have an acceptable shelf life of a mixed product that contains probiotic bacteria. Enteral solutions go through a thermal sterilization or are aseptically filled in presterilized containers, thus killing or removing any live bacteria added during the production process. If the bacteria are added directly into the solution during the production/filling process and after sterilization, the bacteria are likely to be re-activated by the presence of water, and would accordingly multiply and finally die within a few weeks or months after production, often much before the product is actually used. The metabolites of the bacteria might also change the solution taste and nutritional value.

To avoid the interaction between the solution and the bacteria prior to ingestion, special delivery systems have been integrated into solution containers like e.g., Tetrabrik or Pet bottles (see, for example, PCT application PCT/US98/21490). Since these delivery systems are more or less an integral part of the packaging, the producer cannot choose during or after production to have some of the products have the delivery system and some not to have it.

There have been a number of devices designed to add particulate components to liquid systems, particularly with beverages to which a component is to be added just before consumption of the beverage. These devices include tubular devices, such as telescopic packaging infusion units formed as tubes from a liquid impermeable material. For example U.S. Pat. No. 3,102,465 and PCT/AAU97/00680 disclose straw-shaped units that can be opened so that the ingredient contained in the unit can be dispensed. A number of patents, for example, U.S. Pat. Nos. 4,860,929 and 4,986,451, provide tubular devices closed on both ends and having perforations along the sides to allow granular material to be released and dissolved in contact with water or another solvent. Other methods of adding a material to a liquid by means of a straw-device include coating the outside of one end of a straw with a flavored coating that dissolves when the straw is placed in a liquid or making the end of a straw in the form of a spoon made of a soluble substance. Other straw-shaped novelty inventions provide straws with internal or external decorative features and substances.

In the parent application hereto, Ser. No. 09/387,947, an additive tube is provided containing a selected material, such as bacterial cells or other additive, on the inside of the tube. The tube is wrapped and sealed in an outer watertight envelope until time for usage. At the time of usage, the outer envelope is taken away and when the tubular device penetrates a solution container such as a beverage or an enteral solution, the selected material is added to the solution as the solution flows through the tube. The parent application does not specifically provide a particular means of adding bacterial cells to enteral bags.

It is therefore an object of the invention to provide a simple low-cost and consumer-friendly system to protect bacteria for an extended term at room temperature, and have a ready-to-use system for the patient or the user of an enteral bag or similar container after this extended term.

It is a further object to provide a device that enables addition to an enteral bag of an ingredient such as a probiotic microorganism or other oxygen-sensitive or water-sensitive component.

It is a further object of the invention to provide a means of adding probiotic bacteria or other additives to enteral solutions which have been through an aseptic or sterile treatment, e.g., sterile filtration, irradiation or thermal sterilization.

It is a further object of the invention to provide a device for adding components to enteral bags, which is enclosed in a water and moisture tight container until it the device is opened and ready for use.

It is a further object of the invention to provide a means for long-term storage of health promoting bacteria.

It is a further object of the invention to provide a new delivery system for other moisture-sensitive or oxygen-sensitive components, such as certain amino acids, peptides, nucleotides, vitamins, hormones and proteins.

Other objects and advantages will be more fully apparent from the following disclosure and appended claims.

### SUMMARY OF THE INVENTION

The invention herein is an additive tube containing a selected material, such as bacterial cells or other additive, for addition of the selected material to a liquid enteral nutrition product. Additive tubes are separately packaged to reduce exposure of the additive that is within the additive tube to water and oxygen. The additive tube is attached to the rest of the system (the delivery line, other tubes, and enteral bag, when present) just prior to use with a patient.

Other objects and features of the inventions will be more fully apparent from the following disclosure and appended claims.

### BRIEF DESCRIPTION OF THE DRAWINGS

FIG. 1 is a side perspective view of the additive tube of the invention attached at its upper end to a standard fitting on the end of a tube extending from an enteral bottle, and at its lower end to a standard fitting on a tube that delivers the liquid enteral nutrition product to a patient.

FIG. 2 is a partial exploded side-perspective view of the additive tube of FIG. 1 and the ends of the tubing to which the additive tube is connected.

FIG. 3 is a cross-section of a portion of the additive tube that has an adherent selected material inside the tube.

FIG. 4 is a partial view of a partially sectioned additive tube end showing the location of adherent selected material.

FIG. 5 is a perspective view of a packaged additive tube of the invention.

### DETAILED DESCRIPTION OF THE INVENTION AND PREFERRED EMBODIMENTS THEREOF

The invention herein provides a device for adding living cells of a probiotic microorganism to a liquid enteral nutrition product dispensed from any type of enteral container, such as an enteral bottle or enteral bag. The enteral nutrition product is administered to a patient through tubing. The device of the invention comprises a liquid impermeable additive tube having an open bore. The open bore is surrounded by an inner tube wall and extends from an open upper end of the tube to an open lower end of the tube. There is a hollow adaptor at each open end of the additive tube for connecting the additive tube to tubing attached either to the enteral container or extending from the additive tube to tubing leading to the patient. There is a coating material on a portion of the inner tube wall, preferably either an oil or wax or a combination of both depending on the desired release-rate in a particular product. The coating holds, or contains, a suspension of the living cells within the open bore adherent to the inner tube wall. The additive tube is packaged in a flexible, essentially water vapor tight envelope enclosing the tube for long-term storage and to protect the living cells of the probiotic microorganism from moisture. The living cells of the probiotic microorganism or other selected additive may be removed from the inner tube wall and mixed with the liquid enteral nutrition product by allowing the liquid to flow from the enteral container flow through the tubing connected to the additive tube and the open bore to the patient.

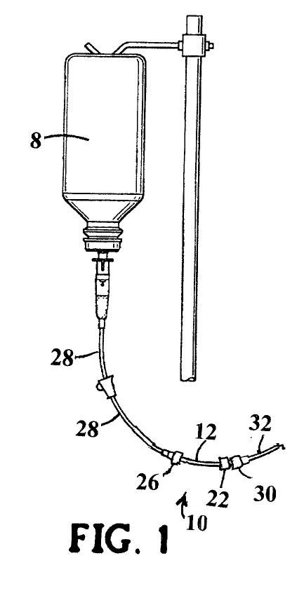

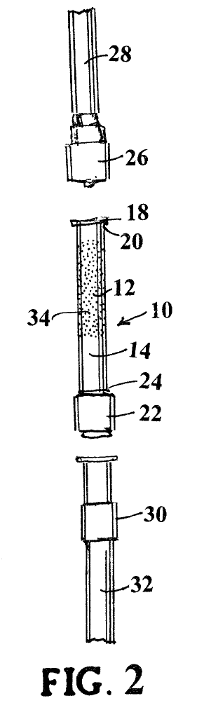

As shown in FIGS. 1-2, the device 10 of the present invention for adding a selected additive to a liquid container 8 comprises a liquid impermeable additive tube 12 having an open bore 14 that is surrounded by an inner tube wall 16 and extends from a hollow upper adapter 18 at an open upper end 20 of the additive tube 12 to a hollow lower adapter 22 at an open lower end 24 of the additive tube 12. Even with the adapters 18, 22 attached, additive tube 12 remains open at both ends. Tube 12 preferably has dimensions ranging from a width of about 3-15 mm and a length of about 10-100 mm in size and is preferably formed from a synthetic polymeric material such as polyethylene or polypropylene. Additive tube 12 may be other than straight if desired for a particular purpose, so long as the adapters 18, 22 on the end are able to connect as described herein and liquid is able to flow through additive tube 12 from the enteral bag or bottle to the patient, carrying with it the additive. Preferably, adapter 18 is structured so that it may be connected to a standard connector 26 on tubing 28 coming from an enteral nutrition liquid source 8 and adapter 22 is structured so that it may be connected to a standard connector 30 on tubing 32 for standard enteral nutrition liquid delivery to a patient (FIG. 1). The additive tube 12 of the invention may be used with alternatively designed enteral nutrition apparatuses by utilizing appropriately designed adapters at each end of additive tube 12 which can connect to the connectors and tubing of the alternative apparatuses.

In the preferred embodiment of the invention herein, a selected material 34, such as a suspension of typically probiotic microorganism, for example, lactic bacteria or bifidus bacteria, is added to the additive tube 12 as described below. In the preferred embodiments of the invention, during production of the tube of the invention, a filling injector delivers the selected material into the open bore of the additive tube 12 as is known in the art (not shown; see co-pending Ser. No. 09/387,947). The inner tube wall 16 is then either sprayed with a special solution containing the selected additive by allowing for nearly full surface coverage or a defined volume of droplets is delivered to cover part of the inner tube wall 16. The tube material must also be able to withstand the suspension media used for the bacteria or whatever other selected material is used.

It is important that additive tube 12 have the capability to hold the suspension, and to hinder the suspension from unintentional leakage out of additive tube 12. This is accomplished either by surface tension through appropriate selection of the material of which additive tube 12 is made or by treatment of additive tube 12. It also could be done by altering the viscosity of the suspension such as by means known in the art.

Although not required for the invention, in the preferred embodiments of the invention, the inner wall 16 of the additive tube 12 is coated or is otherwise surface-modified to give a higher surface tension. Thus, in the preferred embodiments, the invention further includes a coating material 36 for holding a suspension of the selected material 34 within the open bore 14 adherent to the inner tube wall 16. This coating material 36 may comprise any coating substance which is non-toxic to humans and to the bacteria to be added to the tube, and which adheres to the inner tube wall 16, for example, a dietary oil such as corn oil or a wax. In preferred composition the coating material is a mixture of dietary oil and fat in order to get the desired viscosity that will both hold the additive material in the tube during storage but also release it at a proper rate when the tube is used. In this preferred composition the coating material is also mixed with the additive material. The coating material 36 may be applied in a number of ways, for example, by insertion of an elongated stick or other device coated with the substance, with a tubular filling device, or by spraying the inside of tube 12.

The bacteria or other additive may be applied with the coating material 36, or be separately applied to selected positions inside the tube (for example, a small drop of about 10 μl) evenly sprayed all over the inside of the tube after coating the inside of tube 12. The pattern and extent of distribution of the additive inside tube 12 may be determined by the nature of the additive and the intended use. Thus, for an easily soluble additive, even distribution is less critical. If the additive dissolves quickly and is at the distal end of tube 12, it may be desirable to position the additive close to the distal end, so that the additive becomes quickly dispersed in the solution in the container. To minimize exposure of an oxygen-sensitive additive, the additive may be placed in the inside tube 12 in the form of a droplet, which would have less surface area than an evenly dispersed material inside the tube 12.

Preferably the selected material 34 that is to be dispensed from the additive tube 12 comprises lyophilized cells of one or more probiotic microorganisms, such as various Lactobacillus or Bifidobacteria strains. Depending on the type of additive being added to tube 12, the suspension containing the additive may need to specially treated to optimize shelf stability and appropriate retention in, and release from, tube 12. For bacteria, the suspension containing the bacteria should not contain a significant amount of water, and should be fairly resistant to oxidation. The suspension should dissolve or release or carry the additive, such as bacteria, into the fluid that is being consumed, at typical use temperatures (e.g., 0° C.-40° C.). Thus, for a number of additives such as bacterial additives, the dried additive is preferably granulized into a very fine powder to insure solubility and even distribution in the solution.

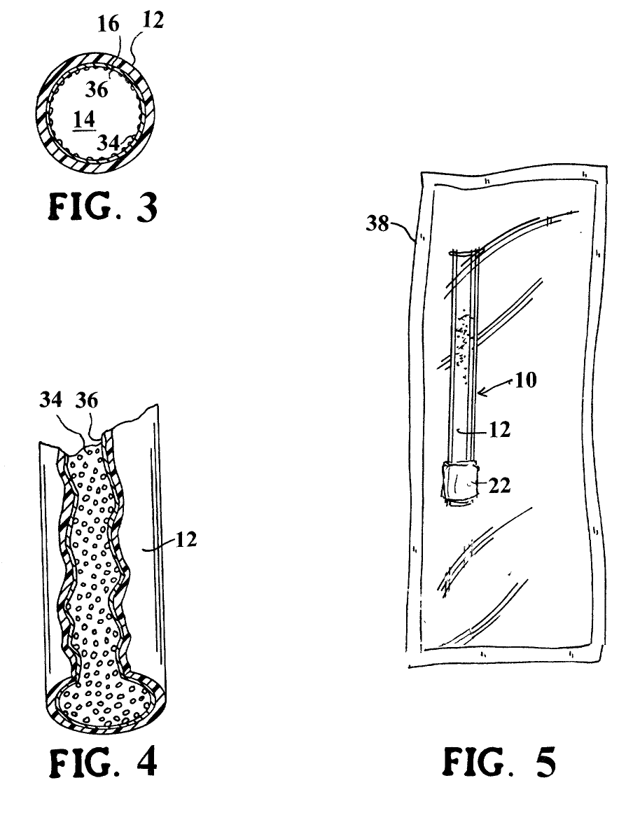

The bacterial suspension or other additive to be contained in the device is preferably prepared in a sufficiently concentrated formulation so that surface tension/adhesion withholds the suspension in the tube. The concentration of the suspension is optimized to give a good ratio between volume and number of bacteria per ml. Preferably the concentration of bacteria is not lower then 1% in the suspension. The cells are preferably mixed directly into the suspending liquid under a nitrogen protective flow to reduce vapor and oxygen presence. Although cells of bacteria (selected material 34) would not be visible to the naked eye, the selected material 34 is depicted in the figures as small dots (FIGS. 2 and 5), or as small bumps or circles (e.g., in FIGS. 3 and 4, respectively), to show their position.

Other selected materials that could be added to liquids using the device of the invention include vitamins, colorants, minerals, trace elements, homeopathic medicines, drugs, enzymes and the like.

Although the selected material 34, such as bacterial cells, may be added by hand to the tube 12 by providing a second tube having a smaller outer diameter than the inner diameter of the liquid impermeable tube as described in the parent application hereto, the addition procedure is preferably automated for purposes of efficiency. In either case, preferably the selected material 34 is added to an additive tube 12 that has been treated in part or entirely with the coating material 36.

As shown in FIG. 5, the additive tube 12 is then placed into a water-resistant outer envelope 38 enclosing the additive tube 12 using a packaging machine as is known in the art. Preferably the packaging process is done under a nitrogen flow, or other gas with negligible or no water and oxygen content to reduce the water and oxygen level in the outer envelope 38, and any head space in the packaging should be limited as is known in the art. The materials used in the manufacture of the invention, in particular, the additive tube 12 and the outer envelope 38, must be capable of protecting the bacteria or other selected material from contamination, oxygen and moisture for periods of up to 12 months storage at room temperature. The outer envelope 38 must be substantially impermeable to water vapor and should have sufficient flexibility and toughness to prevent unintentional puncture, and is preferably made of flexible polymeric material or an aluminum foil, coated on a polymeric film. In all cases the envelope construction is made in such a way that when sealed, water and moisture are prevented from entering the additive tube 12. The envelope material should also be easy to tear open at the point of use. The envelope 38 is preferably made of a polyolefinic material coated with aluminum or of a synthetic polymer as is known in the art with a low water permeation rate. A preferred material for the outer envelope 38 is a polyethylene or polypropylene, including both homopolymers and copolymers of these polymer families, with an aluminum layer as an outer layer. If a transparent envelope 38 is desired, the polyethylene/polypropylene structure may have outer layer comprising a polymer of ethyl vinyl alcohol or polyvinylidene chloride. Alternatively, polyethylene and/or polypropylene may be used without an aluminum layer if there is not a need for protection from oxygen. Those of ordinary skill in the art may substitute other suitable packaging material.

To use the additive tube 12, it is removed from envelope 38, and attached to an enteral tube of a selected patient by attaching the hollow adapters 18, 22 at the corresponding tube ends 20, 24 to connectors 26, 30 on tubing 28 and 32, respectively. Once the additive tube 12 is attached, liquid from the enteral bottle or bag flows through the tubing 28 above the additive tube 12, through the additive tube 12, and through the tubing 32 below the additive tube 12 and to the patient. The bacteria (or other selected additives) are integrated into the solution, giving a desirable dose of additive in the product.

While the invention has been described with reference to specific embodiments, it will be appreciated that numerous variations, modifications, and embodiments are possible, and accordingly, all such variations, modifications, and embodiments are to be regarded as being within the spirit and scope of the invention.

What is claimed is:

1. A device for adding living cells of a probiotic microorganism to a liquid enteral nutrition product dispensed from an enteral container, said enteral nutrition product being administered to a patient through tubing, said device comprising:
(a) a liquid impermeable additive tube having an open bore, said bore being surrounded by an inner tube wall and extending from an open upper end of the tube to an open lower end of the tube;
(b) a hollow adaptor at the upper open end for connecting the additive tube to tubing attached to the enteral container;
(c) a hollow adaptor at the lower open end of the additive tube for connecting the additive tube to tubing leading to the patient;
(d) a coating material on a portion of the inner tube wall, said coating material selected from the group consisting of oils and waxes and holding a suspension of the living cells within the open bore adherent to the inner tube wall; and
(e) a flexible, essentially water vapor tight envelope enclosing the tube for long-term storage and to protect the living cells of the probiotic microorganism from moisture;
wherein the living cells of the probiotic microorganism may be removed from the inner tube wall and mixed with the liquid enteral nutrition product by allowing the liquid to flow from the enteral container flow through the tubing connected to the additive tube and through the open bore to the patient.

2. The device of claim 1, wherein the probiotic microorganism comprises Lactobacillus.

3. The device of claim 1, wherein the selected material comprises a suspension of lyophilized live bacteria.

4. The device of claim 3, in which the concentration of live bacteria at the point of use is at least 1% of the suspension when the device is manufactured.

5. The device of claim 1, wherein the viscosity of the suspension is modified to provide maximum adherence of the suspension to the device.

6. The device of claim 1, wherein the tube is made from a synthetic polymer material.

7. The device of claim 1, wherein the tube has a diameter of about 3-15 mm and a length of about 10-100 mm.

8. The device of claim 1, wherein the open bore of the tube has been surface modified to give a higher surface tension.

9. The device of claim 1, wherein the envelope is made of a polyolefinic material coated with aluminum.

10. The device of claim 9, wherein the envelope is made of a synthetic polymer with a low water permeation rate.

11. The device of claim 1, wherein the suspension of material is distributed along the full length of the inner tube wall.

12. The device of claim 1, wherein the suspension of material is primarily placed at the lower end of the tube.

13. The device of claim 1, wherein the living cells of the probiotic microorganism are removed from the inner tube wall at a particular release rate.

14. A method of making a device for dispensing a living cells of a probiotic microorganism into a liquid enteral nutrition product dispensed from an enteral container, said enteral nutrition product being administered to a patient through tubing, comprising:
(a) providing a liquid impermeable additive tube having an open bore, said bore being surrounded by an inner tube wall and extending from an open upper end of the tube to an open lower end of the tube, and a hollow adapter at each end of the additive tube;
(b) coating a portion of the inner tube wall within the open bore with a coating material selected from the group consisting of oils and waxes and holding a suspension of the living cells of the probiotic microorganism so that the coating material containing the living cells of the probiotic microorganism adheres to the inner tube wall; and
(c) providing a flexible, essentially water vapor tight envelope enclosing the tube for long-term storage and to protect the living cells of the probiotic microorganism from moisture;
wherein the living cells of the probiotic microorganism may be removed from the inner tube wall and mixed with the liquid enteral nutrition product by allowing the liquid to flow from the enteral container flow through the tubing connected to the additive tube and through the open bore to the patient.

15. The method of claim 14, wherein the inner tube wall is coated with the selected material by insertion of a second tube that has a coating of the selected material into the lower end of the additive tube.

16. The method of claim 14, wherein the probiotic microorganism comprises Lactobacillus.

17. The method of claim 14, wherein the selected material comprises a suspension of lyophilized live bacteria.

18. The method of claim 17, in which the concentration of live bacteria at the point of use is at least 1% of the suspension when the device is manufactured.

19. The method of claim 14, wherein providing the liquid impermeable additive tube comprises providing a tube made from a synthetic polymer material.

20. The method of claim 14, wherein providing a liquid impermeable additive tube comprises providing a tube having a diameter of about 3-15 mm and a length of about 10-100 mm.

21. The method of claim 14, further comprising surface-modification of the open bore of the tube to give a higher surface tension.

22. The method of claim 21, wherein providing the flexible, essentially water vapor tight envelope enclosing the tube comprises providing a polyolefinic material coated with aluminum.

23. The method of claim 14, wherein providing the flexible, essentially water vapor tight envelope enclosing the tube comprises providing an envelope made of a synthetic polymer with a low water permeation rate.

24. The method of claim 14, wherein coating a portion of the inner tube wall comprises distributing the suspension of material along the full length of the inner tube wall.

25. The method of claim 14, wherein coating a portion of the inner tube wall comprises distributing the suspension of material at the lower end of the tube.

26. The method of claim 14, further comprising modifying the viscosity of the suspension to provide maximum adherence of the suspension to the device.

27. The device of claim 14, wherein the living cells of the probiotic microorganism are removed from the inner tube wall at a particular release rate.
